(19) United States
(12) Patent Application Publication
(10) Pub. No.: US 2019/0071429 A1
(43) Pub. Date: Mar. 7, 2019

(54) MULTIPLE-FUNCTIONAL PROBE AND USES THEREOF

(71) Applicant: Institute of Nuclear Energy Research, Atomic Energy Council, Executive Yuan, R.O.C., Taoyuan (TW)

(72) Inventors: Cheng-Liang PENG, Taoyuan (TW); Tsai-Yueh LUO, Taoyuan (TW); Ying-Hsia SHIH, Taoyuan (TW)

(21) Appl. No.: 15/988,344

(22) Filed: May 24, 2018

(30) Foreign Application Priority Data
Sep. 1, 2017 (TW) 106130037

(51) Int. Cl. C07D 403/14; G01N 33/574; A61K 51/04

(52) U.S. Cl. CPC C07D 403/14; C07B 2200/05; A61K 51/0482; G01N 33/574

(57) ABSTRACT

Disclosed herein are a multi-functional probe and uses thereof. The multi-functional probe has a main structure represented by chemical Formula (1), and is configured to diagnose and treat the cancers.

### CROSS-REFERENCE TO RELATED APPLICATION

This application claims priority to Taiwan Patent Application No. 106130037 filed in the Taiwan Patent Office on Sep. 1, 2017, the entire content of which is incorporated herein by reference.

### TECHNICAL FIELD

The present disclosure relates to the technical field of multi-functional probes, and more particularly to a multi-functional probe useful in the treatment and diagnosis of cancers.

### BACKGROUND

In recent years, malignant tumors ranked the 1^{st} place in the top ten leading causes of morbidity. The five-year survival rate can be significantly improved, if the cancers can be diagnosed in an early stage of development and the patients are properly treated as early as possible. With the increase in the population suffering from cancers worldwide, development of drugs for the diagnosis and treatment of cancers becomes extremely important in the biopharmaceutical industry.

At present, the treatment for primary or metastatic tumors in clinic is, in principle, mainly surgical resection; however, operation is impractical in many cases. Therefore, several topical therapies are developed. Among them, thermal tumor ablation is confirmed to be effective and safe. Thermal tumor ablation therapy is a first-line low invasive treatment mode when the tumor patient is not suitable for receiving operation.

The greatest challenge encountered in the thermal tumor ablation therapy is how to improve the targeting to a site to be treated and reduce the injury to a normal tissue. Photothermal therapy (PTT) achieves an effect of ablating the tumor tissue by generating heat in the tumor by means of a special light source. With the addition of a photosensitizer, the sensitivity of the tumor tissue to light of a specific wavelength can be enhanced, to accomplish the purpose of treating a subject. However, in the prior art, the thermal ablation therapy is limited by the image monitoring during and after the treatment, since synchronous monitoring cannot be realized.

In view of this, there is an urgent need in the art for an improved probe for treatment and diagnosis, to overcome the disadvantages existing in the prior art.

### SUMMARY

To facilitate the understanding of the fundamental meaning of the present disclosure, brief description of the present disclosure is provided in the summary, which is not a complete description of the present disclosure and not intended to define the technical features or scope of the present invention.

An aspect of the present disclosure relates to a multi-functional probe having a structure represented by Formula (1):

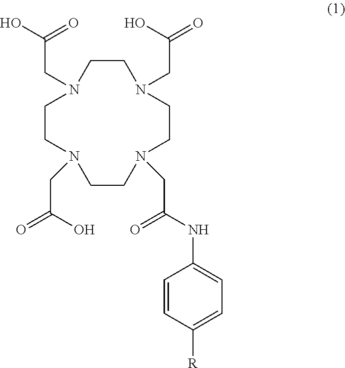

where R is

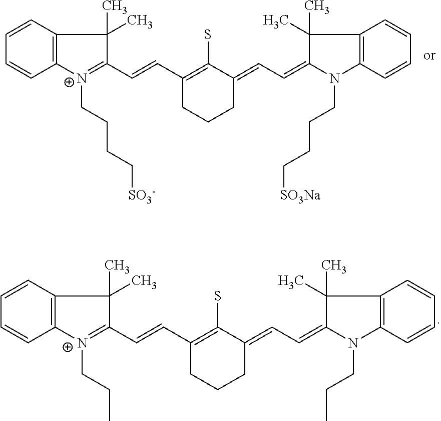

According to a specific embodiment of the present invention, in the compound of Formula (1) of the present invention, R is

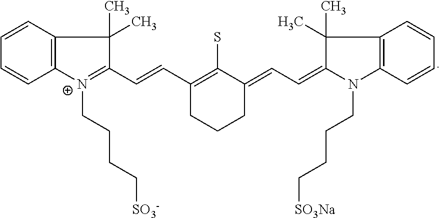

According to another specific embodiment of the present invention, in the compound of Formula (1) of the present invention, R is

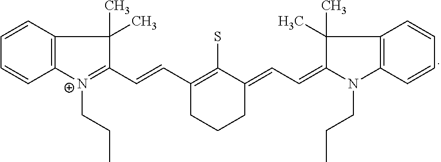 [SMILES: CCCN1/C(=C/C=C2\CCCC(/C=C/C3=[N+](CCC)c4ccccc4C3(C)C)=C2S)C(C)(C)c2ccccc21]

According to other embodiments of the present invention, the multi-functional probe of the present invention further comprises a radioactive isotope labeled on the compound of Formula (1). In an optional embodiment, the radiation isotope is rhenium-188, technetium-99 m, indium-111, lutetium-177, gallium-68, yttrium 90, flurine-18, copper-64 or gadolinium.

Another aspect of the present invention relates to a contrast agent. Particularly, the contrast agent comprises a multi-functional probe according to any of the above embodiments and an excipient acceptable in the contrast agent.

Another aspect of the present invention relates to use of the multi-functional probe according to any of the above embodiments in the preparation of drugs for diagnosing or treating cancers. In an optional embodiment, the cancer is selected from the group consisting of leukemia, lymphoma, diaphyseal osteosarcoma, multiple myeloma, testicular cancer, thyroid cancer, prostate cancer, throat cancer, cervix cancer, nasopharyngeal carcinoma, breast cancer, colorectal cancer, pancreatic cancer, gastric cancer, head and neck cancer, esophageal cancer, rectal cancer, bladder cancer, kidney cancer, lung cancer, liver cancer, brain cancer, melanoma, squamous cell carcinoma or skin cancer.

Another aspect of the present invention relates to use of the multi-functional probe according to any of the above embodiments in the diagnosis or treatment of subjects with or suspected of having cancers.

The central concept, the technical means employed and the various implementations of the present invention can be fully understood by those of ordinary skill in the art from reading the following embodiments.

### BRIEF DESCRIPTION OF DRAWINGS

The foregoing and other objects, features, advantages and embodiments of the present invention will become more apparent from the following brief description of drawings, in which:

FIG. 1 is a flow chart of a process for producing the present multi-functional probe DOTA-NIR790 according to an embodiment of the present invention;

FIG. 2 is a flow chart of a process for producing the present multi-functional probe DOTA-NIR780 according to an embodiment of the present invention;

FIG. 3A is a single photon emission computed tomography (SPECT) image of an animal model of subcutaneous tumor administered with the present multi-functional probe Indium-111-DOTA-NIR790 according to an embodiment of the present invention;

FIG. 3B is a single photon emission computed tomography (SPECT) image of an animal model of subcutaneous tumor administered with the present multi-functional probe Indium-111-DOTA-NIR780;

FIG. 3C shows results of near-infrared fluorescence (NIRF) imaging of an animal model of subcutaneous tumor administered with the present multi-functional probe Indium-111-DOTA-NIR790 according to an embodiment of the present invention;

FIG. 3D shows results of NIRF imaging of an animal model of subcutaneous tumor administered with the present multi-functional probe Indium-111-DOTA-NIR780 according to an embodiment of the present invention;

FIG. 4A shows a NanoSPECT/CT image of an animal model of brain metastatic tumor administered with the present multi-functional probe Indium-111-DOTA-NIR790 according to an embodiment of the present invention, in which a result of imaging the whole body of the mice is shown on the left, and a local acquisition result of the head is shown on the right;

FIG. 4B shows results of NIRF imaging of an animal model of brain metastatic tumor administered with the present multi-functional probe Indium-111-DOTA-NIR790 according to an embodiment of the present invention;

FIG. 4C shows results of NIRF imaging of the brain tissue in an animal model of brain metastatic tumor administered with the present multi-functional probe Indium-111-DOTA-NIR790 according to an embodiment of the present invention;

FIG. 5A is a bar diagram showing the biodistribution of the present multi-functional probe Indium-111-DOTA-NIR790 in an animal model of colorectal cancer according to an embodiment of the present invention;

FIG. 5B is a bar diagram showing the biodistribution of the present multi-functional probe Indium-111-DOTA-NIR790 in an animal model of colorectal cancer according to an embodiment of the present invention;

FIG. 5C is a bar diagram showing the biodistribution of the present multi-functional probe Indium-111-DOTA-NIR790 in an animal model of head and neck cancer according to an embodiment of the present invention; and

FIG. 5D is a bar diagram showing the biodistribution of the present multi-functional probe Indium-111-DOTA-NIR790 in an animal model of lung cancer according to an embodiment of the present invention.

FIG. 6A is a diagram showing the measurement results of the temperature of the tumor tissue according to an embodiment of the present invention.

FIG. 6B is a diagram showing the measurement results of the tumor volume according to an embodiment of the present invention.

### DETAILED DESCRIPTION OF DISCLOSED EMBODIMENTS

To make the description of the present disclosure more elaborate and complete, the following description of the implementations and specific embodiments of the present invention will be described in further detail; however, the implementations and specific embodiments of the present invention are not limited thereto.

Unless otherwise indicated, the scientific and technical terms used herein have the same meanings as those understood by those of ordinary skill in the art. Moreover, the terms used herein cover the singular and plural referents, unless otherwise specified.

The term “subject” or “patient” refers to an animal that is capable of receiving the thermosensitive carrier of the present invention. In a preferred embodiment, the animal is a mammal, and in particular human.

The “cancer” may be a non-solid tumor or a solid tumor. For example, the cancer may include, but is not limited to, leukemia, lymphoma, diaphyseal osteosarcoma, multiple myeloma, testicular cancerthyroid cancer, prostate cancer, throat cancer, cervix cancer, Nasopharyngeal carcinoma, breast cancer, colorectal cancer, pancreatic cancer, gastric cancer, head and neck cancer, esophageal cancer, rectal cancer, bladder cancer, kidney cancer, lung cancer, liver cancer, brain cancer, melanoma, squamous cell carcinoma, or skin cancer.

As used herein, the term “about” usually means that the actual value is within 10%, 5%, 1%, or 0.5% of a particular value or range, and that the actual value is within the acceptable standard error of the mean value, depending on the considerations of those of ordinary skill in the art to which this present invention pertains. Besides the experimental examples, or unless otherwise expressly stated, the ranges, the amounts, the values and the percentages used herein are modified with “about.” Therefore, unless otherwise stated, the values or parameters disclosed in this specification and the appended claims are all approximate value and may vary depending on the requirements.

To solve the problems existing in the prior art, the present inventors have initially proposed a multifunctional single probe molecule, which is different from the probes in the prior art in that the probe of the present invention has the capabilities of tumor diagnosis by means of near infrared fluorescence and nuclear medical imaging, photothermal tumor treatment, and targeting radiotherapy with isotope. Specifically, the structure of the compound of the present invention consists essentially of two portions, one portion of which is an infrared fluorescent dye, that is, a heptamethine cyanine dye, which has a unique optical property of strong absorption in the near-infrared band and a tumor targeting performance, can enhance the sensitivity of tumor tissue to a light source of specific wavelength, and can achieve the effect of ablating a tumor tissue by producing heat in the tumor after excitation with a special light source; and the other portion of which is a chelating group (e.g., DOTA) which is labeled with a radioactive isotope for radiotherapy.

Various examples are provided below for illustrating various different implementations of the present invention, so that the technical teachings of the present invention can be practiced by those skilled in the art to which the present invention pertains in accordance with the disclosure herein. Thus, the following examples are not to be construed as limiting the scope of the present invention, and all references cited herein are hereby expressly incorporated by reference in their entirety as part of this specification

### Example 1. Synthesis of Multi-Functional Probe of the Present Invention

1.1. Synthesis of DOTA-NIR790

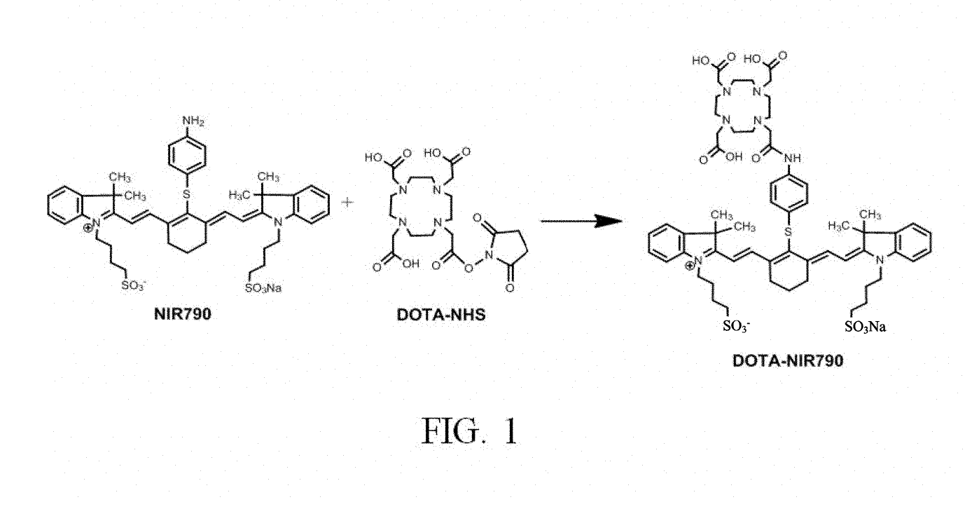

The main process for chemical synthesis in this example was shown in FIG. 1. The synthesis steps were as follows. NIR-790 (2-[2-[2-(4-aminobenzenethio)-3-[(1,3-dihydro-3,3-dimethyl-1-(4-sulfobutyl)-2H-indol-2-ylidene)-ethylidene]-1-cycloxen-1-yl]-ethynyl]-3,3-dimethyl-1-(4-sulfobutyl)-3H-indolium, innersalt, monosodium) (83.8 mg, 100 μmol) was dissolved in anhydrous DMF (5 ml), and triethyl amine (20 mg, 200 μmol) was added. Then, a solution of DOTA-NHS (153 mg, 200 μmol) dissolved in DMF (1 ml) was added to the reaction mixture, and stirred for 3 days at room temperature. The obtained crude product was purified by HPLC on a C-18 column using 60% CAN and 40% H2O containing 0.1% TFA as a mobile phase, to obtain a pure target product. A green solid (21 mg, 17.2%) was obtained after drying, and the structure of the multi-functional probe of the present invention was determined after analysis by HPLC and identification by nuclear magnetic resonance spectrometry and mass spectrometry.

1.2. Synthesis of DOTA-NIR780

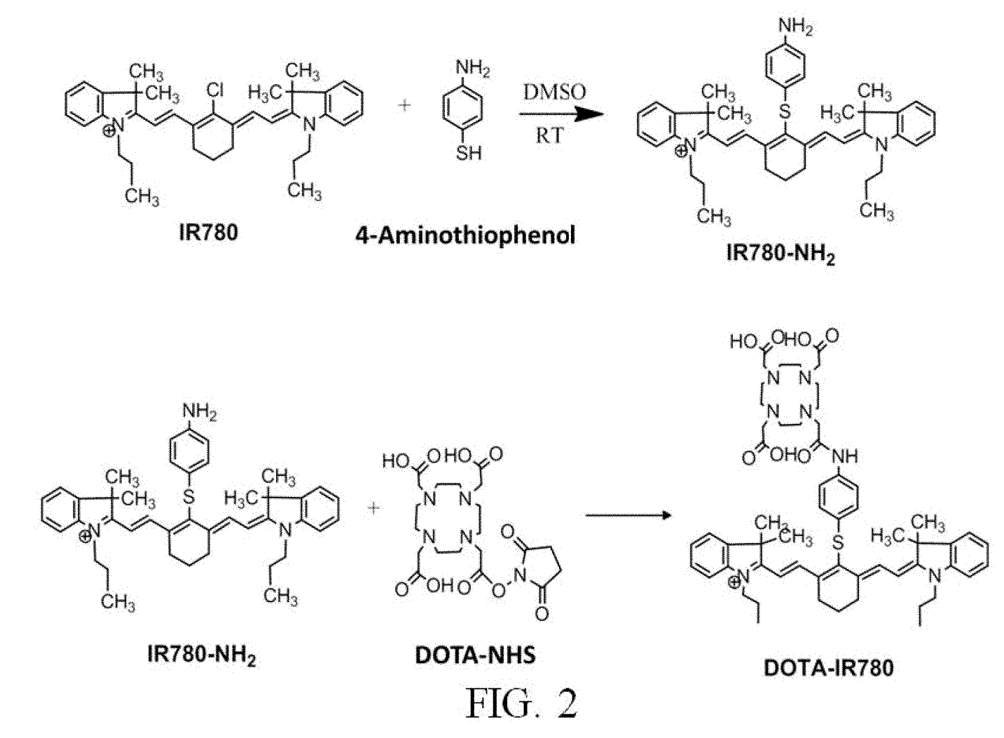

The main process for chemical synthesis in this example was shown in FIG. 2. The synthesis steps were as follows. IR780 iodide (2-[2-[2-Chloro-3-(1,3-dihydro-3,3-dimethyl-1-propyl-2H-indol-2-ylidene) ethylidene]-1-cyclohexen-1-yl]ethenyl]-3,3-dimethyl-1-propylindolium iodide) (120 mg, 143.2 μmol) and 4-aminothiophenol (300 mg, 958 μmol) were dissolved in anhydrous DMF (5 ml), and reacted overnight at room temperature. The obtained crude product was purified by preparative HPLC coupled with a C-18 column, to obtain a pure target product IR780-NH2. A green solid (120 mg, 79.4%) was obtained after drying, which was then analyzed by HPLC, and identified by nuclear magnetic resonance spectrometry and mass spectrometry. IR780-NH2 (75.5 mg, 100 μmol) was dissolved in anhydrous DMF (5 ml), and triethyl amine (20 mg, 200 μmol) was added. Then, a solution of DOTA-NHS (153 mg, 200 μmol) dissolved in DMF (1 ml) was added to the reaction mixture, and stirred for 3 days at room temperature. The obtained crude product was purified by preparative HPLC coupled with a C-18 column using, as a mobile phase, 60% CAN and 40% H₂O containing 0.1% TFA which was gradient to 100% ACN in 15 min, to obtain a pure target product. A green solid (21 mg, 17.2%) was obtained after drying, and the structure of the multi-functional probe of the present invention was determined after analysis by HPLC and identification by nuclear magnetic resonance spectrometry and mass spectrometry.

### Example 2. Preparation of Multi-Functional Probe Labeled with Radioactive Isotope: Indium-111-DOTA-NIR790 or Indium-111-DOTA-NIR780

¹¹¹InCl₃ (activity 370) was added to 0.2 M sodium acetate buffer (300 μl, pH 5.5) containing DOTA-NIR790 or DOTA-NIR780 (1 mg), and reacted for 1 hr at 37° C. with agitation. After reaction, Indium-111-DOTA-NIR790 (or DOTA-NIR780) was adsorbed onto RP-18 column, and purified by washing with physiological saline and eluting with ethanol. The radiochemical purity of Indium-111-DOTA-NIR790 (or DOTA-NIR780) was evaluated by Radio-HPLC, and was shown to be up to 95% or higher after purification.

### Example 3. Use of the Multi-Functional Probe of the Present Invention in the Diagnosis and Treatment of Cancers

3.1. Establishment of Animal Models

3.1.1 Establishment of Animal Model of Subcutaneous Tumor

The experimental animals used in this experimental example were female BALB/c nude mice (5 to 6 weeks old), and the breast cancer 4T1 (ATCC® CRL-2539™) cells (1×10⁶) were inoculated subcutaneously to the mice at the flank of the right and left legs. During experiment, the tumor size and the body weight were measured periodically once every three days. The tumor volume was calculated by a formula: πab²/6, where a was the length of the tumor and b was the width of the tumor. When the tumor volume reached about 150-200 mm³, subsequent tests were carried out.

3.1.2. Establishment of Animal Model of Brain Metastatic Tumor

The experimental animals used in this experimental example were female BALB/c mice (8 weeks old). Before the implantation of the tumor, the mice were anesthetized through exposure to 1% to 3% isoflurane. The 4T1-luc breast cancer cells (2×10⁴) were suspended in PBS (2 μL), and slowly injected at a depth that was 3.7 mm from the dura mater. The duration of the process was 3 min. The needle was left in place for 5 min, and then slowly withdrawn. The wound on the head was sutured with a 6-0 suture. 10 days after the animal model receiving the inoculation with the cancer cells, subsequent tests were carried out.

3.1.3. Establishment of Animal Model of Human Colorectal Cancer

The experimental animals used in this experimental example were female BALB/c mice (8 weeks old). The HCT-116 colorectal cancer cells (3×10⁶) were suspended in PBS (100 μL), and subcutaneously injected at a site between the thigh and the back. 14 days after the animal model receiving the inoculation with the cancer cells, subsequent tests were carried out.

3.1.4. Establishment of Tumor Animal Model of Human Head and Neck Cancer

The experimental animals used in this experimental example were female SCID mice (8 weeks old). The FaDu head and neck cancer cells (5×10⁶) were suspended in PBS (100 μL), and subcutaneously injected at a site between the thigh and the back. 21 days after the animal model receiving the inoculation with the cancer cells, subsequent tests were carried out.

3.1.5. Establishment of Tumor Animal Model of Human Lung Cancer

The experimental animals used in this experimental example were female SCID mice (8 weeks old). The A549 lung cancer cells (3×10⁶) were suspended in PBS (100 μL), and subcutaneous injected at a side of the chest. 21 days after the animal model receiving the inoculation with the cancer cells, subsequent tests were carried out.

3.1.6. Establishment of Animal Model of Mouse Colorectal Cancer

The experimental animals used in this experimental example were female BALB/c mice (8 weeks old). The CT26 colorectal cancer cells (1×10⁶) were suspended in PBS (100 μL), and subcutaneously injected at a site between the thigh and the back. 14 days after the animal model receiving the inoculation with the cancer cells, subsequent tests were carried out.

3.2. Biodistribution Assay of the Multi-Functional Probes Indium-111-DOTA-NIR790 and Indium-111-DOTA-NIR780 of the Present Invention in the Animal Model of Subcutaneous Tumor

In this experimental example, single photon emission computed tomography (SPECT) and near-infrared fluorescence (NIRF) imaging were performed. The in-vivo distribution of the multi-functional probe labeled with a radioactive isotope (that is, Indium-111-DOTA-NIR790 or Indium-111-DOTA-NIR780) in the animal model of subcutaneous tumor was evaluated.

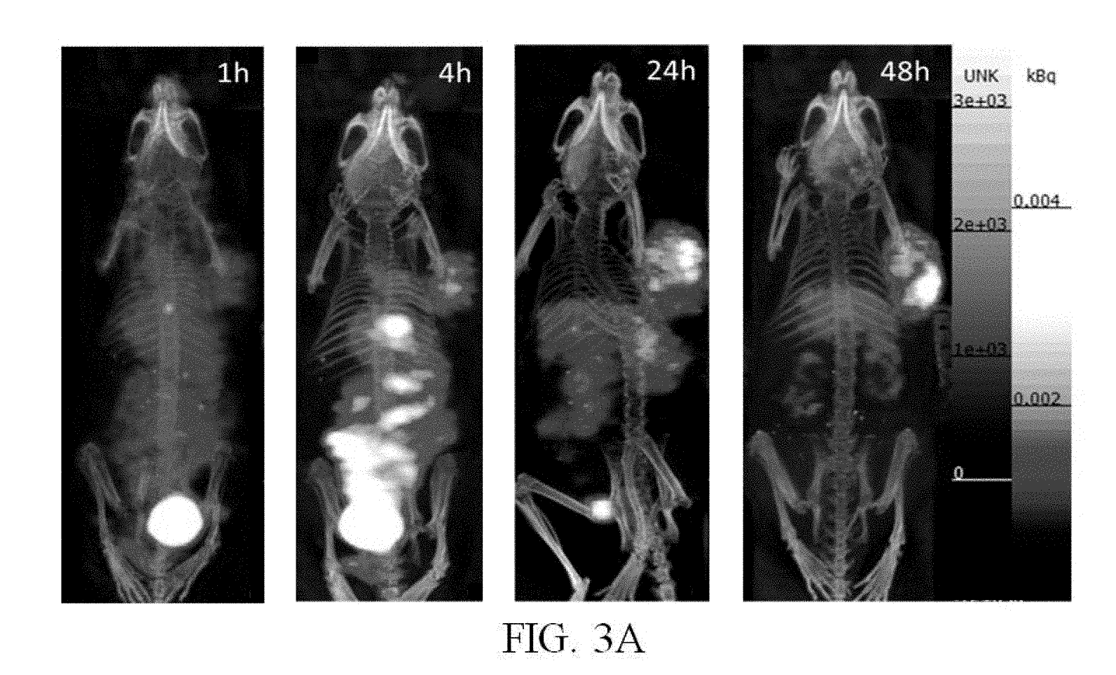

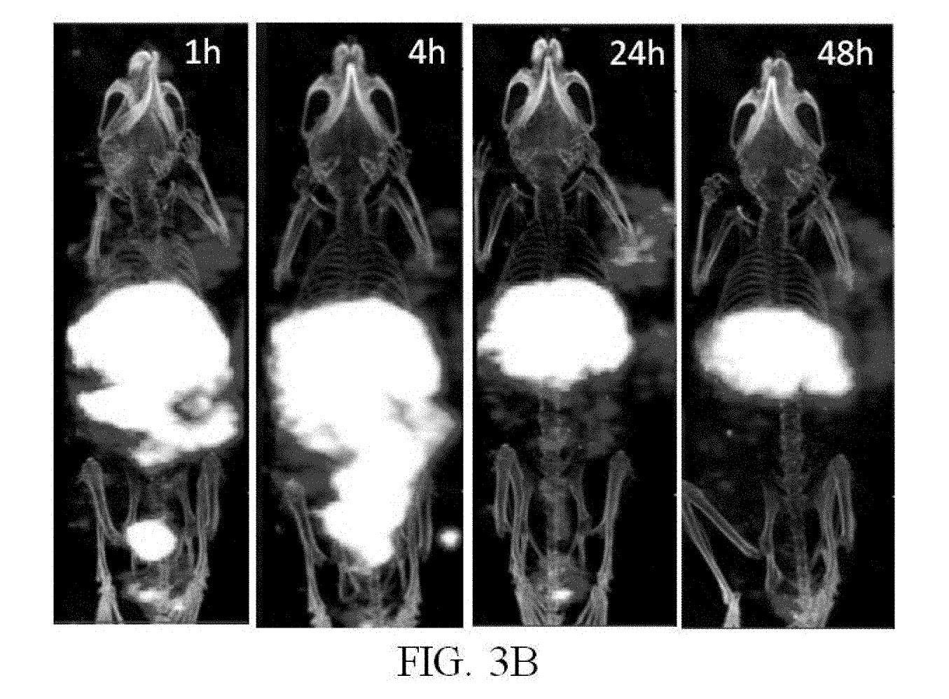

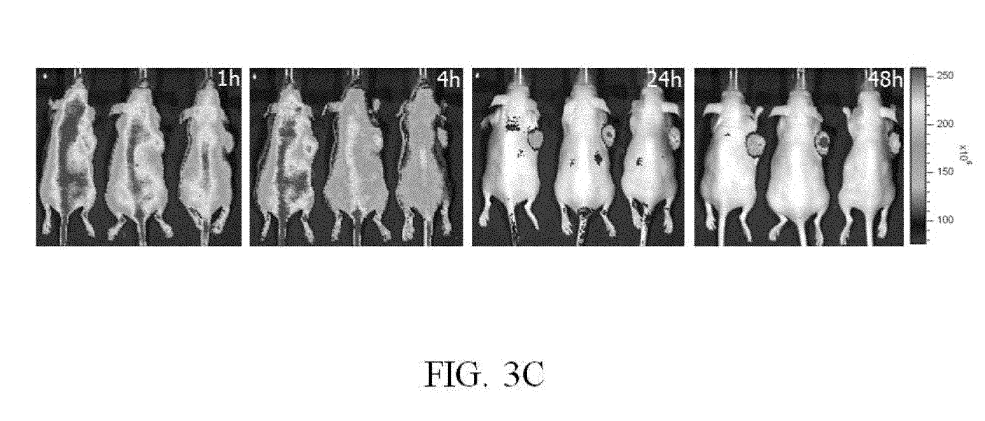

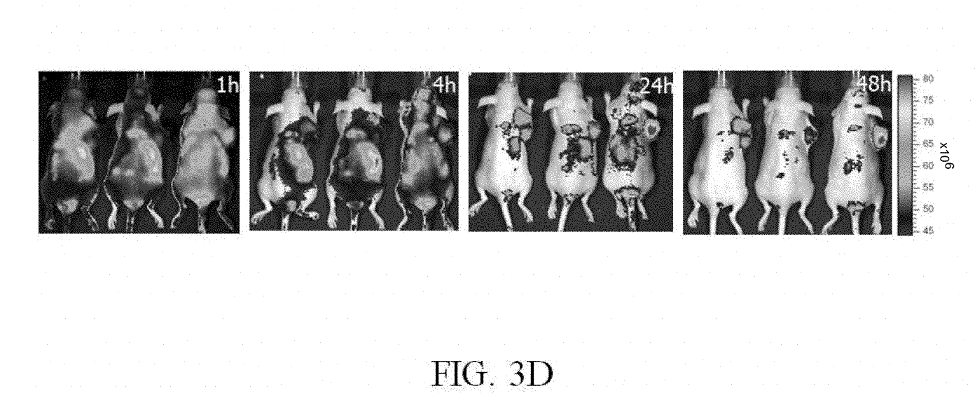

Indium-111-DOTA-NIR790 and Indium-111-DOTA-NIR780 (about 37 MBq of Indium-111) were respectively intravenously injected to the animal model of subcutaneous tumor obtained in Example 3.1.1, and then imaged by NanoSPECT/CT. The in-vivo images of the multi-functional probe of the present invention in mice were acquired at 1, 4, 24 and 48 hrs. The mice were sacrificed and the organs were collected and quantitatively and qualitatively analyzed by a γ-counter and by photoradiography. The results are shown in FIG. 3A and FIG. 3B. For example, as shown in FIG. 3A, 24 hrs after injection, the multifunctional probe indium-111-DOTA-NIR790 of the present invention was largely accumulated in the tumor site of the mice (1.78±0.37% ID/g), and the amount accumulated at the tumor site at 48 hours is still up to 1.67±0.21% ID/g. In addition, the drug is easy to metabolize, and will not accumulate in other organs. Moreover, 24 hrs after injection, the multi-functional probes Indium-111-DOTA-NIR790 and Indium-111-DOTA-NIR780 of the present invention have an accumulation ratio in tumor/muscle of 12.84±0.65 and 2.97±0.96 respectively, indicating that the multi-functional probe of the present invention is accumulated in the tumor site much more greatly than in the muscle tissue.

Additionally, for the NIRF imaging, the multi-functional probes Indium-111-DOTA-NIR790 and Indium-111-DOTA-NIR780 (about 100-300 μg DOTA-NIR790) of the present invention were respectively intravenously injected to the animal model of subcutaneous tumor, and then quantified by taking pictures and imaging at day 1, 4, 24 and 48 hours using an IVIS imaging system at ex 710-760 nm/em 810-875 nm (ICG filter set). The results are shown in FIG. 3C and FIG. 3D. As shown, the multi-functional probe Indium-111-DOTA-NIR790 has a result of NIRF imaging that is in agreement with the result of SPECT imaging, indicating that the multi-functional probe of the present invention is specific for tumors.

3.2. Biodistribution Assay of the Multi-Functional Probe Indium-111-DOTA-NIR790 of the Present Invention in the Animal Model of Brain Metastatic Tumor

In this experimental example, SPECT and NIRF imaging were performed. The in-vivo distribution of the multi-functional probe labeled with a radioactive isotope (that is, Indium-111-DOTA-NIR790) in the animal model of brain metastatic tumor was evaluated.

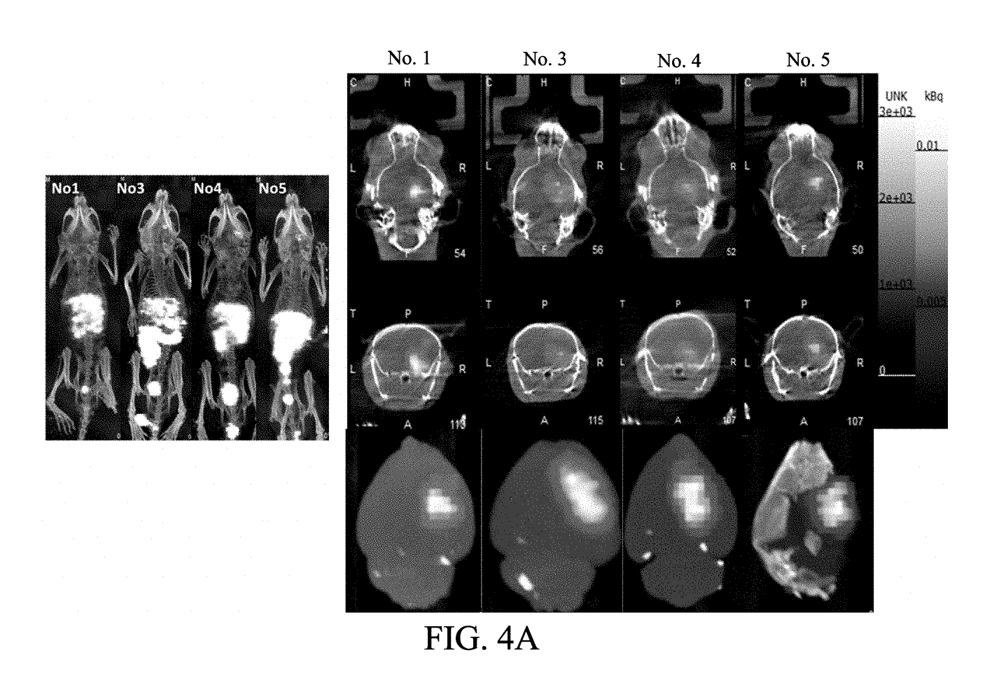

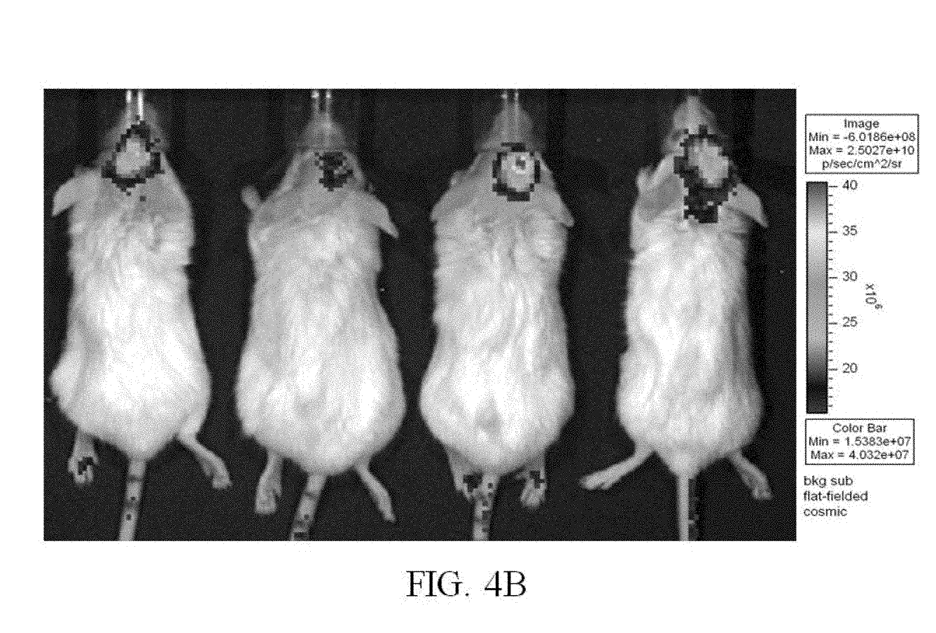

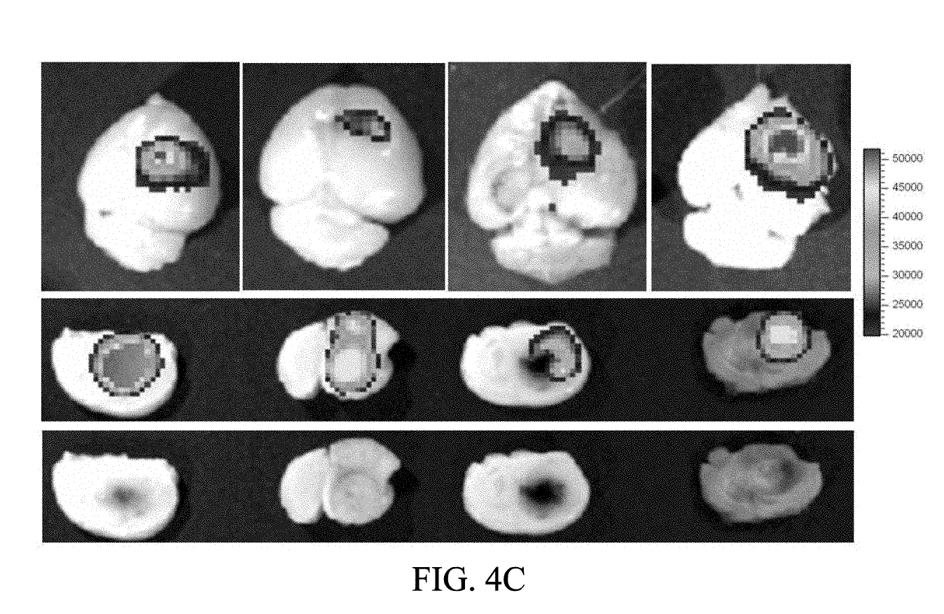

Indium-111-DOTA-NIR790 (about 37 MBq of Indium-111) was intravenously injected to the animal model of brain metastatic tumor obtained in Example 3.1.2, and then imaged by NanoSPECT/CT. The in-vivo images of the multi-functional probe of the present invention in mice were acquired. The mice were sacrificed, and the brain tissue was collected and quantitatively and qualitatively analyzed by a γ-counter and by photoradiography. The results are shown in FIG. 4A. Additionally, for the NIRF imaging, the multi-functional probe Indium-111-DOTA-NIR790 (about 100-300 μg DOTA-NIR790) of the present invention was intravenously injected to the animal model of brain metastatic tumor, and then quantified by taking pictures and imaging using an IVIS imaging system at ex 710-760 nm/em 810-875 nm (ICG filter set). The mice were sacrificed, and the brain tissue was collected and analyzed as described above. The results are shown in FIG. 4B and FIG. 4C. It can be known from the results that the multi-functional probe of the present invention can similarly specifically bind to brain tumor tissues, and similar results are achieved in the SPECT and NIRF imaging.

3.2. Biodistribution Assay of the Multi-Functional Probe Indium-111-DOTA-NIR790 of the Present Invention in Animal Models of Other Cancers

In this experimental example, SPECT imaging was performed. The in-vivo distribution of the multi-functional probe labeled with a radioactive isotope of the present invention (that is, Indium-111-DOTA-NIR790) in various animal models of cancers obtained in 3.1.3 to 3.1.6 was evaluated.

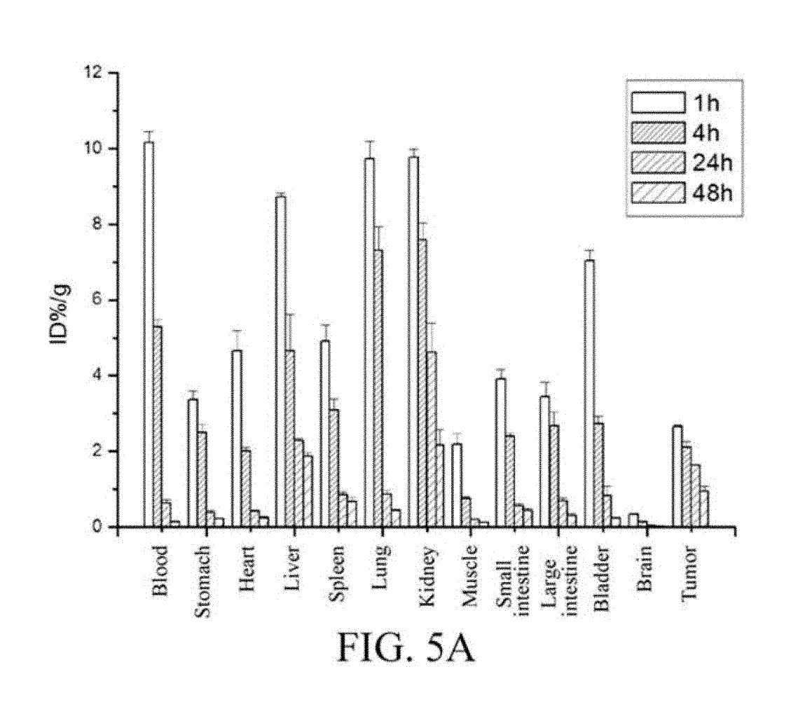

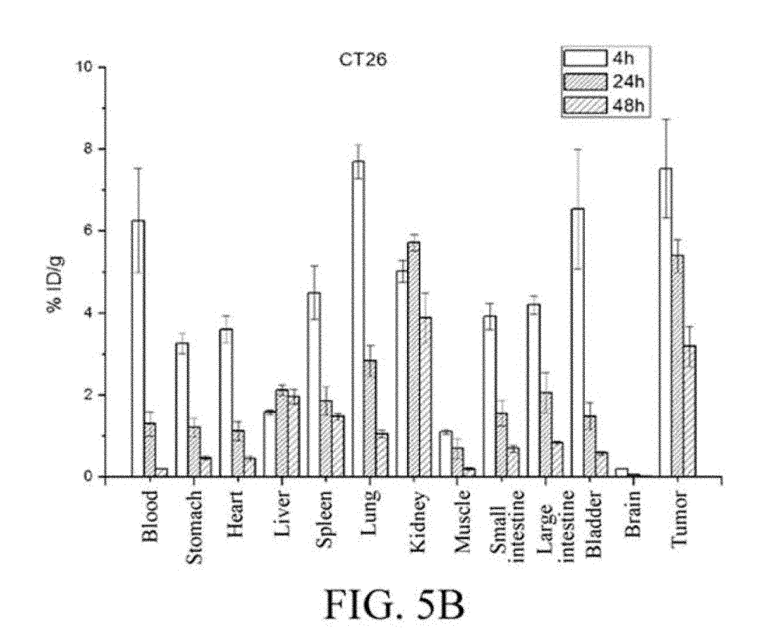

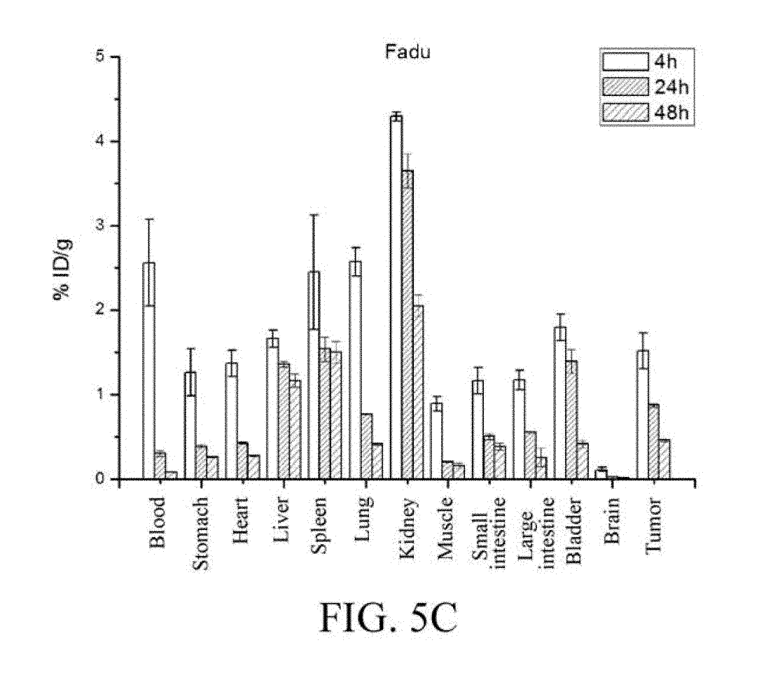

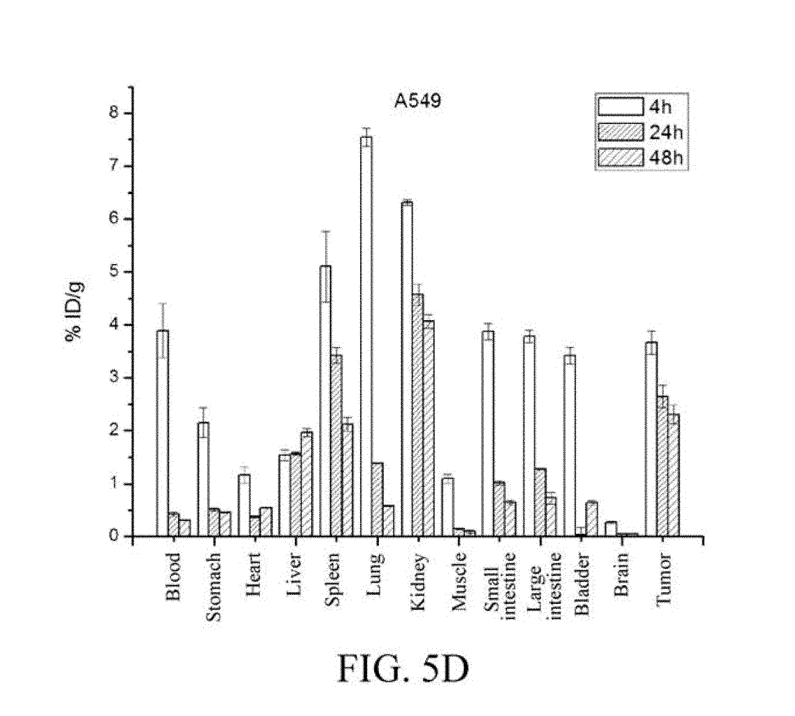

After the animal models of cancers were each intravenously injected with Indium-111-DOTA-NIR790 (about 37 MBq of Indium-111), the radioactivity was determined at 1, 4, 24 and 48 hrs. The results are shown in FIGS. 5A to 5D.

As shown by the biodistribution result in the mouse model of human colorectal cancer (HCT-116), the amount accumulated 24 hrs and 48 hrs after injection is 1.62±0.29% and 0.94±0.15% ID/g respectively, and the accumulation ratio in tumor/muscle at 48 hrs is 7.66±1.13. As shown by the biodistribution result in the mouse model of mouse colorectal cancer (CT26), the amount accumulated 24 hrs and 48 hrs after injection is 5.39±0.40% and 3.19±0.49% ID/g respectively, and the accumulation ratio in tumor/muscle at 48 hrs is 15.18±2.13. As shown by the biodistribution result in the mouse model of human head and neck cancer (FaDu), the amount accumulated 24 hrs and 48 hrs after injection is 0.87±0.02% and 0.46±0.02% ID/g respectively, and the accumulation ratio in tumor/muscle at 48 hrs is 4.27±0.19. As shown by the biodistribution result in the mouse model of human lung cancer (A549), the amount accumulated 24 hrs and 48 hrs after injection is 2.65±0.21% and 2.31±0.15% ID/g respectively, and the accumulation ratio in tumor/muscle at 48 hrs is 18.98±3.35.

In summary, it can be known from the above results that the multi-functional probe of the present invention can be accurately accumulated in the tumor site from the systemic circulation of the animals, confirming that the multi-functional probe provided in the present invention can be used in combination with the near infrared fluorescence and nuclear medical imaging to provide an efficacy in the diagnosis and treatment of cancers and/or tumors.

### Example 4. Effect of the Multi-Functional Probe of the Present Invention on Photothermal Therapy of Colorectal Cancer

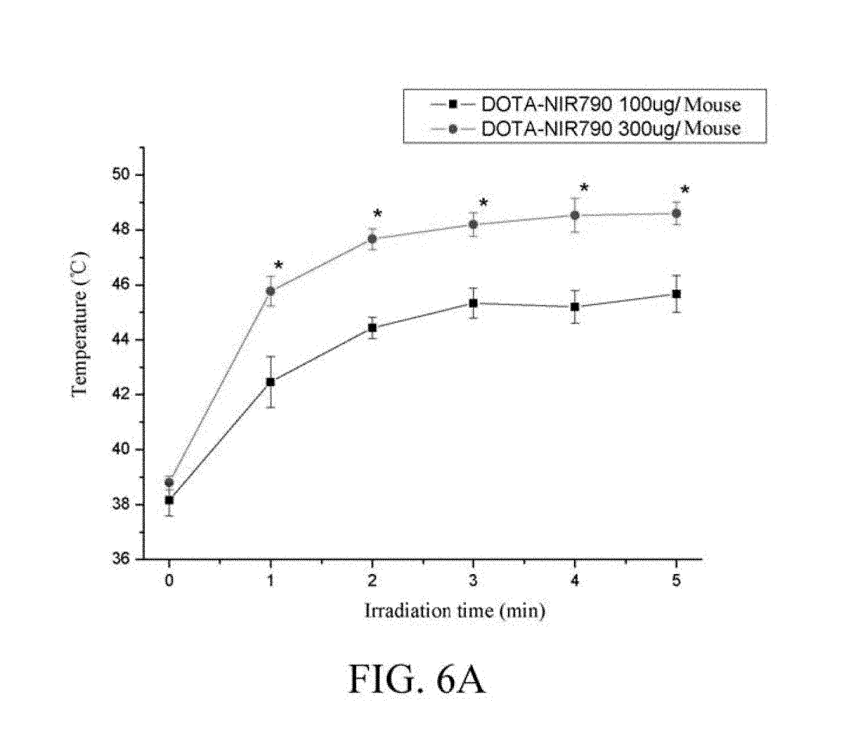

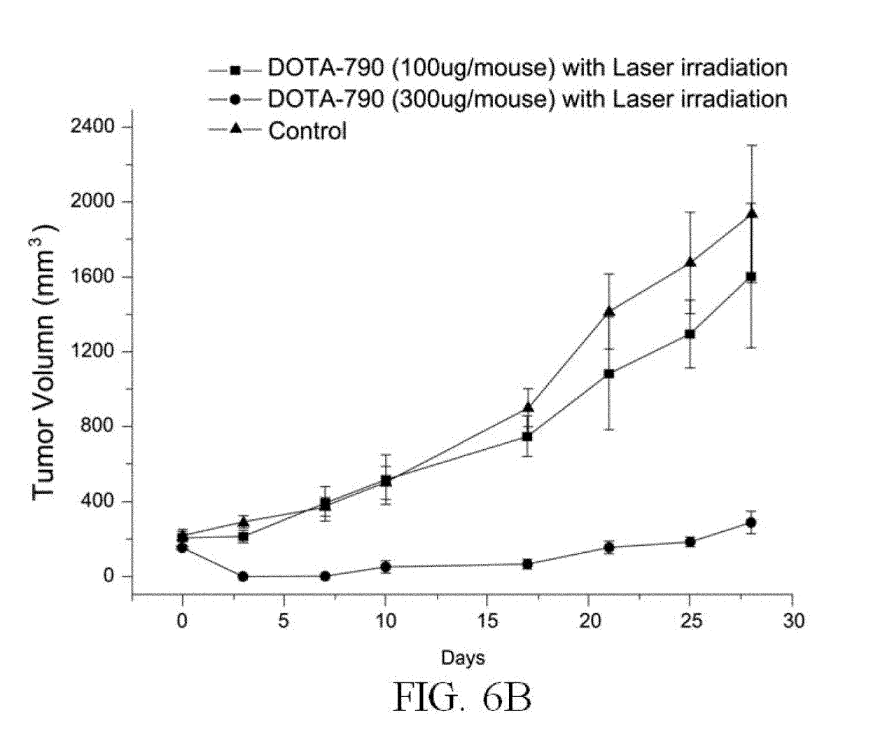

A labeled multi-functional probe for diagnosis and treatment of tumors (DOTA-NIR790, about 100˜300 μg) was administered to the tumor animal model of HCT-116 obtained in Example 3.1.3. 24 hrs after injection, the animals were irradiated with laser at 808 nm. The measurement results of the temperature of the tumor tissue are shown in FIG. 6A. Moreover, the measurement results of the tumor volume are shown in FIG. 6B. It can be known from the results shown in FIG. 6A that due to the optical property of strong absorption in the near-infrared band, the multi-functional probe of the present invention is effective for photothermal therapy by producing heat in the tumor tissue. Furthermore, as shown by the results shown in FIG. 6B, the tumor volume is effectively controlled and the tumor growth is effectively inhibited in the group administered with a high concentration (300 μg) of multi-functional probe of the present invention.

The specific examples disclosed above are not intended to limit the scope of the claims of the present invention, and modifications may be made by those skilled in the art based on their general knowledge without departing from the principle and spirit of the present invention. Therefore, the scope claimed by the present invention is as defined by the claims of the present invention.

What is claimed is:

1. A multi-functional probe, having a structure represented by Formula (1): 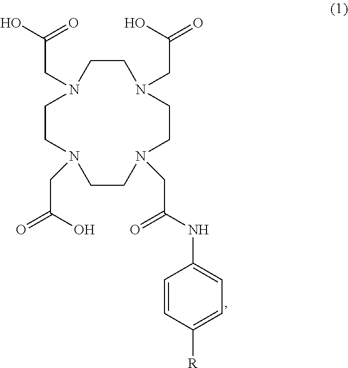
wherein R is

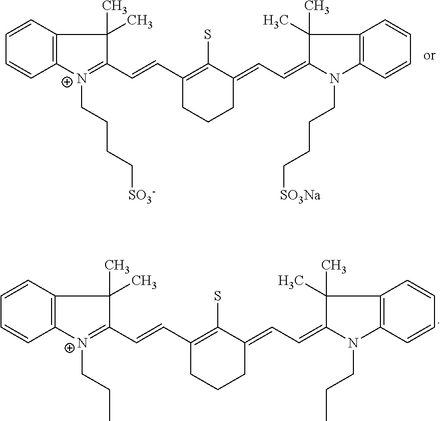

2. The multi-functional probe according to claim 1, wherein R is

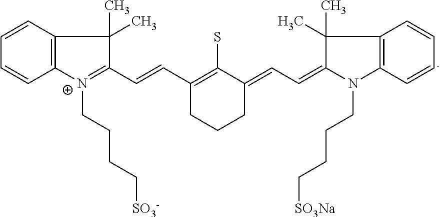

3. The multi-functional probe according to claim 1, wherein R is

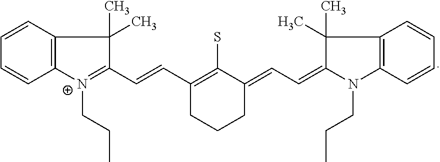 [SMILES: CCCN1/C(=C/C=C2\CCCC(/C=C/C3=[N+](CCC)c4ccccc4C3(C)C)=C2S)C(C)(C)c2ccccc21]

4. The multi-functional probe according to claim 1, further comprising a radioactive isotope labeled on the compound of Formula (1).

5. The multi-functional probe according to claim 4, wherein the radioactive isotope is rhenium-188, technetium-99 m, indium-111, lutetium-177, gallium-68, yttrium 90, flurine-18, copper-64 or gadolinium.

6. A contrast agent comprising:
the multi-functional probe according to claim 5; and
an excipient acceptable in the contrast agent.

7. A method for diagnosing or treating a subject having or suspected of having cancers, comprising the step of administering an effective amount of the multi-functional probe according to claim 1 to the subject.

8. The method according to claim 7, wherein the cancer is selected from the group consisting of: leukemia, lymphoma, diaphyseal osteosarcoma, multiple myeloma, testicular cancer, thyroid cancer, prostate cancer, throat cancer, cervix cancer, Nasopharyngeal carcinoma, breast cancer, colorectal cancer, pancreatic cancer, gastric cancer, head and neck cancer, esophageal cancer, rectal cancer, bladder cancer, kidney cancer, lung cancer, liver cancer, brain cancer, melanoma, squamous cell carcinoma, and skin cancer.
